(19) United States
(12) Patent Application Publication
(10) Pub. No.: US 2006/0011220 A1
(43) Pub. Date: Jan. 19, 2006

(54) Shopping cart wash tunnel

(76) Inventor: Barbara A. Mueller, Marblehead, OH (US)

Correspondence Address: HAHN LOESER & PARKS, LLP, One GOJO Plaza, Suite 300, AKRON, OH, 44311-1076, US

(21) Appl. No.: 11/181,342

(22) Filed: Jul. 14, 2005

Related U.S. Application Data
(60) Provisional application No. 60/588,058, filed on Jul. 14, 2004.

(51) Int. Cl. B08B 3/00; B08B 3/04; B08B 3/12

(52) U.S. Cl. 134/45; 134/123; 134/50; 134/61; 134/82; 134/151; 134/198

(57) ABSTRACT

A shopping cart sanitizing apparatus and method is provided that uses a wash tunnel to automatically dispense sanitizing agents onto shopping carts. The wash tunnel uses a conveyor or other means for moving shopping carts into the apparatus, where sanitizing agents are automatically dispensed onto the shopping carts. Suitable sanitizing agents include soap and water, chemical agents, or a combination of disinfecting fluids.

This application claims priority from U.S. provisional application Ser. No. 60/588,058, the disclosure of which is hereby incorporated by reference.

### TECHNICAL FIELD

The present invention relates to an apparatus and method for sanitizing shopping carts.

### BACKGROUND OF THE INVENTION

A shopping cart, and particularly a shopping cart used in a grocery or discount store, will be used many times per day. With each use, there is a significant opportunity for contamination of the cart with germs that can lead to the spread of disease. Examples of such diseases may include the common cold and influenza viruses, as well as food-borne illnesses including escherichia coli and salmonella.

A typical shopping cart has at least one basket for carrying items, four wheels to transport the cart, and a handle to push or pull the cart. Many carts have a child seat facing the handle. Shopping carts often nest together so that multiple carts can be moved and stored together.

A shopping cart encounters several sources of germs, and the contamination opportunities are centered on several parts of the cart. A primary area of contamination is the handle. First, the customer who is handling food products and merchandise continually touches the handle. Second, child passengers face the handle when placed in the child seat that is conventional on many shopping carts. These child passengers grasp the handle and are also known to bite or drool on the handle. A second area of contamination is the child seat itself, which may be contaminated by the child passengers. A third area of contamination is the basket of the cart. Uncooked meat and poultry items, unwashed fruits and vegetables and plant items are commonly transported in this portion of the shopping cart. Furthermore, the carts do not remain in the store at all times, and they are exposed to a number of contaminates in every trip to the store parking lot. Examples of such contaminates include petroleum based pollutants from automobile exhaust, factory emissions from surrounding industrial sites, road dirt, droppings from birds and other grime.

As shopping cart contamination is a public health concern, there have been some efforts in the field of shopping cart sanitation. Some recent United States patents and published patent applications have focused almost exclusively on the handle. For example, U.S. Pat. No. 5,215,319 to Farris, U.S. Pat. No. 6,065,764 to Moseley, and U.S. published application 2004/0021279 to Sobo all provide sanitary covers for the handle. U.S. Pat. No. 6,129,417 to Cohen-Fyffe teaches a seat cover for the shopping cart child seat area. Additionally, various retailers provide disinfectant wipes to their customers. Some commercial cleaning service companies offer cart cleaning to the retailer using portable pressure washers. However, seat and handle covers become dirty from the cart and must be replaced or washed after each use. Wipes are costly and only locally effective if used properly. Not all customers use covers and wipes, and not all retailers can afford to offer them. The effectiveness of commercial cleaning services varies with the operator. Additionally, commercial cleaning services may be infrequently used, allowing germs and grime to build up. While all of these efforts to reduce the spread of disease via shopping carts have furthered the art of cart sanitation, none of the known prior art addresses sanitation of the entire cart in a regular and convenient way. There remains a need for a convenient and thorough cleaning system and method for sanitizing shopping carts.

### SUMMARY OF THE INVENTION

In response to the foregoing concerns, the present invention provides a method and apparatus for sanitizing shopping carts. One embodiment of the cart sanitizing system comprises a wash tunnel, having a first end and a second end. The wash tunnel has a means for conveying the shopping carts from the first end to exit through the second end. The present invention further includes means for detecting the position of the shopping carts within the wash tunnel, and an array of nozzles connected to at least one source of fluid for sanitizing the shopping carts. The apparatus includes means for controlling the array of nozzles; wherein the fluid sprays from the array of nozzles when the shopping carts convey under the array of nozzles.

The wash tunnel includes components designed to effectively sanitize shopping carts as the shopping carts pass through the wash tunnel. In one embodiment, the wash tunnel comprises a soap spray, rinse spray, a dryer, and optionally, disinfectant spray. The method of sanitizing shopping carts comprises propelling a plurality of shopping carts through a wash tunnel through a soap spray and a rinse spray. The method may further comprise propelling the shopping carts through a disinfectant spray and a continuous stream of drying air.

In an alternate embodiment, the shopping carts remain stationary while being sanitized. In this alternate embodiment, the wash tunnel comprises a spray gantry that moves along gantry tracks positioned inside the wash tunnel so that the spray gantry travels over the shopping carts as it moves along the gantry tracks inside the wash tunnel. The spray gantry comprises means for discharging spray of various solutions. The spray gantry may be programmed to operate in a wash sequence, so that soap spray is dispensed as the spray gantry passes over the shopping carts in a first pass. The spray gantry discharges a rinse spray in a second pass over the shopping carts. Optionally, the spray gantry discharges a disinfectant spray in a third pass over the shopping carts. As the shopping carts are moved out of the wash tunnel, they encounter a stream of drying air.

In alternate embodiments, the cart sanitizing system may include a wash tunnel installed in a cart entrance of a retail building. The wash tunnel includes a means for discharging a disinfectant spray. Shopping carts pass through the disinfectant spray as the shopping carts enter the building. Means for conveying shopping carts can be employed to keep the cart operator away from the disinfectant spray.

In other alternate embodiments, the cart sanitizing system includes a steam cleaning apparatus.

The following description sets forth in detail certain illustrative embodiments of the invention, these being indicative of but a few of the various ways in which the principles of the present invention may be employed.

### BRIEF DESCRIPTION OF THE DRAWINGS

FIG. 1 is a side perspective view of a first embodiment of the cart sanitizing system according to the present invention;

FIG. 2 is an enlarged perspective view of a soap spray nozzle of a first embodiment of the cart sanitizing system;

FIG. 3 is an enlarged perspective view of a rinse spray nozzle of the first embodiment of the cart sanitizing system;

FIG. 4 is an enlarged perspective view of a disinfectant spray nozzle of the first embodiment and a third embodiment of the cart sanitizing system;

FIG. 5 is a side perspective view of a second embodiment of the cart sanitizing system according to the present invention;

FIG. 6 is an enlarged perspective view of a spray nozzle on the spray gantry of the second embodiment of the cart sanitizing system;

FIG. 7 is a side perspective view of the third embodiment of the cart sanitizing system;

FIG. 8 is a side perspective view of a fourth embodiment of the cart sanitizing system according to the present invention;

FIG. 9 is an enlarged perspective view of a steam nozzle of the fourth embodiment of the cart sanitizing system;

FIG. 10 is a flow chart of a steam cleaning apparatus of the fourth embodiment of the cart sanitizing system; and

FIG. 11 is a side view of a means for conveying shopping carts according to the fourth embodiment of the present invention.

### DETAILED DESCRIPTION

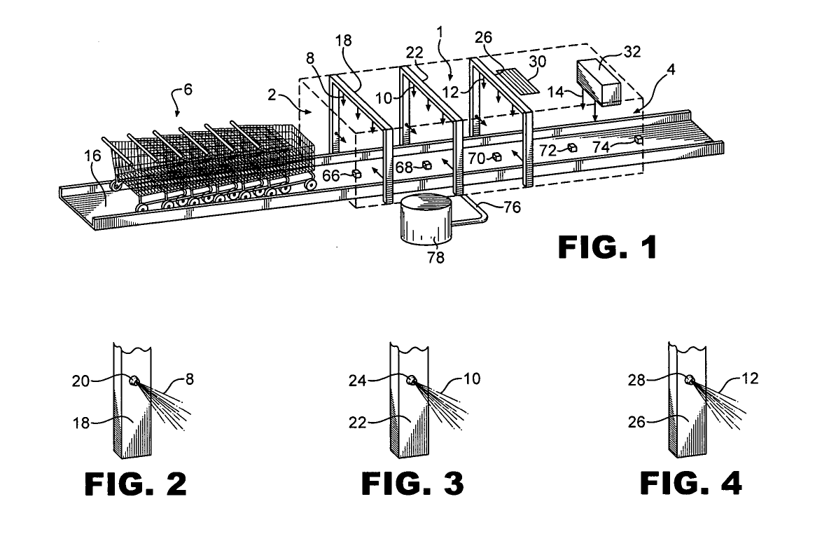

A first embodiment of a wash tunnel 1 is illustrated in FIG. 1. The wash tunnel 1 has a first end 2 and a second end 4 and is sized to accommodate a plurality of shopping carts 6. Shopping carts 6 enter the wash tunnel 1 through the first end 2, travel through the wash tunnel 1 and exit the wash tunnel 1 by passing through the second end 4. The wash tunnel 1 is sized to allow shopping carts 6 to easily pass through the wash tunnel 1. Optionally, the wash tunnel 1 may be large enough for a person to walk through in order to perform maintenance or other operations. The length of the wash tunnel 1 may be sufficient to dispense a soap spray 8, a rinse spray 10, a disinfectant spray 12, and a continuous sheet of high-velocity air 14, each separated a distance sufficient to substantially prevent mixing.

The plurality of shopping carts 6 may be automatically propelled through the wash tunnel 1. Means for conveying the plurality of shopping carts will herein be represented as conveyor 16, and include, but are not limited to a conveyor belt, such as those well known in the art, wherein the belt is made from leather, rubber, wire mesh, chain link, or any other man-made or synthetic material. Another means for conveying includes a guided push/pull chain, and similar configurations well known in the art. Another means for conveying includes a continuous screw shaft, wherein a motor rotates the continuous screw shaft, causing an operably connected push arm to translate along the continuous screw shaft, further discussed below. The use of continuous screw shafts for conveying is well known in the art. Means for conveying the plurality of shopping carts as contemplated herein includes any known device for automatically pulling or pushing the shopping carts 6 through and/or out of the wash tunnel 1.

The conveyor 16 extends longitudinally through the wash tunnel 1 from the first end 2 to the second end 4. In the embodiment illustrated in FIG. 1, the conveyor 16 has a length longer than the length of the wash tunnel 1 such that the conveyor 16 extends beyond the first end 2 and the second end 4 of the wash tunnel 1. In this embodiment, the conveyor 16 moves at a uniform rate, carrying the shopping carts 6 through the wash tunnel 1.

Inside the wash tunnel 1, the shopping carts 6 are conveyed through the soap spray 8 dispensed by a soap delivery apparatus 18. With reference to FIG. 2, the soap delivery apparatus 18 is an aqueous system that dispenses the soap spray 8 from one or more nozzles 20 in a vertical and/or transverse direction. The soap delivery apparatus 18 comprises a source of soap, and further comprises valves, pumps, pipes or tubes, and other traditional and typical fluid handling components all well known in the art, arranged to deliver soap from the source of soap to the nozzles 20. In this embodiment, the soap spray 8 is a high-pressure spray, but in alternate embodiments, the soap spray 8 can be a low-pressure spray or foam.

As used in this written description and in the attached claims, the word “soap” is not limited to traditional salts of fatty acids as used for cleaning. As used herein, “soap” includes natural and synthetic cleaning and wetting soaps, detergents, and surfactants, in a state suitable for spraying.

As used in this written description and in the attached claims, the word “fluid” is not limited to liquid substances. As used herein, “fluid” includes any substance in a liquid, gas or vapor, powder, or other state suitable for spraying.

After the shopping carts 6 pass through the soap spray 8, the shopping carts are conveyed through a rinse spray 10, dispensed by a rinse delivery apparatus 22. The rinse delivery apparatus 22 dispenses rinse spray 10 from one or more nozzles 24 in a vertical and/or transverse direction. The rinse delivery apparatus 22 comprises a source of water or other rinsing agents, and further comprises valves, pumps, pipes or tubes, and other traditional and typical fluid handling components all well known in the art, arranged to deliver the water or other rinsing agents from the source to the nozzles 20. With reference to FIG. 3, the rinse spray 10 is a high-pressure water spray. Alternately, the rinse spray 10 may be a low-pressure spray, and/or may employ chemical rinsing agents.

After passing through the rinse spray 10, the shopping carts 6 preferably continue on the conveyor 16 through the disinfectant spray 12, delivered by a disinfectant delivery apparatus 26. In one embodiment, the disinfectant delivery apparatus 26 dispenses disinfectant spray 12 through one or more nozzles 28, as illustrated in FIG. 4. The disinfectant spray 12 may be a low-pressure spray. Disinfectant spray 12 can be any disinfecting or sanitizing agent suitable for spray application. Disinfecting and sanitizing agents are well known in the art.

The disinfectant delivery apparatus 26 comprises a source of disinfectant, and further comprises valves, pumps, pipes or tubes, and other traditional and typical fluid handling components all well known in the art, arranged to deliver the disinfectant from the source of disinfectant to the nozzles 20.

Vent 30 may be provided to remove disinfectant fumes from the wash tunnel 1 for safe application of the disinfectant spray 12. Alternate embodiments may omit the disinfectant spray 12.

Before exiting the tunnel at the second end 4, the shopping carts 6 are conveyed near a dryer 32. The dryer 32 may be a conventional forced air blower that discharges the continuous sheet of high-velocity air 14 to blow the water off of the shopping carts 6 as they pass by. Such dryers 32 are often called air knives and are well known in the art. Optionally, the continuous sheet of high-velocity air 14 may be heated for faster drying.

One or more of the sprays dispensed in the wash tunnel preferably activates automatically, as described below.

The system disclosed in the first embodiment may be used with a method of sanitizing shopping carts 6. The method comprises propelling a plurality of shopping carts 6 through a wash tunnel 1, passing the shopping carts 6 through an automatically activated soap spray 8, rinse spray 10, disinfectant spray 12, and a continuous sheet of high-velocity air 14.

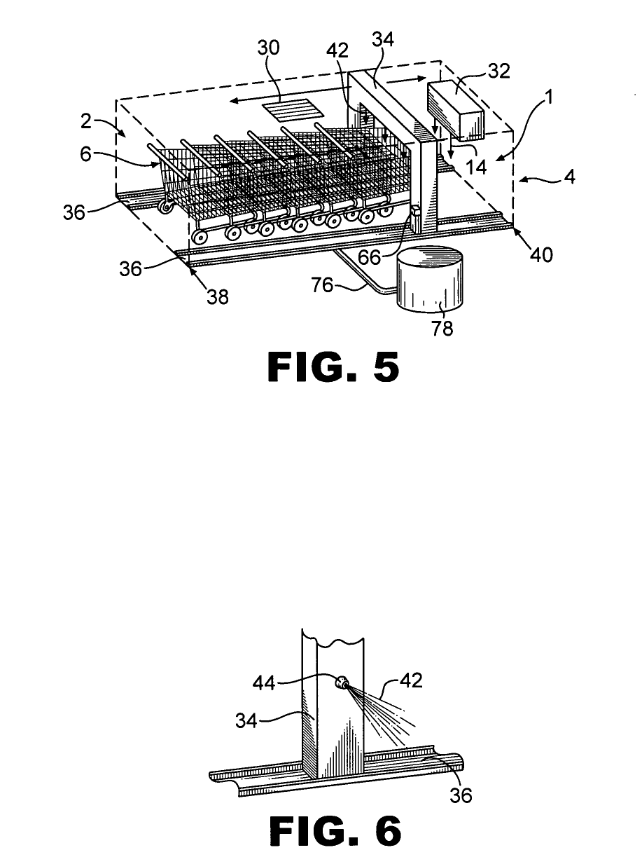

A second embodiment of a wash tunnel 1 is illustrated in FIG. 5. In the second embodiment, the wash tunnel 1 has a first end 2 and a second end 4 and is sized to hold a plurality of shopping carts 6. Shopping carts 6 enter the wash tunnel 1 through the first end 2, travel through the wash tunnel 1 and exit by passing through the second end 4. The wash tunnel 1 length is sufficient to hold a predetermined number of shopping carts 6. The wash tunnel 1 height and width is sized for shopping carts 6 to easily pass through with further space toward the top and sides to accommodate the operation of a spray gantry 34. Gantry tracks 36 extend through the length of the wash tunnel 1 in a direction parallel to the direction of movement of the shopping carts 6. In one embodiment, the gantry tracks 36 are located on the floor, one on the left side and one on the right side. Optionally, the gantry tracks 36 are mounted on the ceiling, or on the sidewalls. If installed on the ceiling, a gantry track 36 may be center mounted. The gantry tracks 36 may have a start position 38 and a return position 40. The spray gantry 34 moves along the gantry tracks 36 from the start position 38 near the first end 2 to the return position 40 near the second end 4. It is contemplated that the start position 38 and return position 40 may be located at any two positions within the wash tunnel 1 or may be at the same position.

The spray gantry 34 comprises means for dispensing spray 42 from one or more nozzles 44. Means for dispensing spray comprises at least one source of fluid, and further comprises valves, pumps, pipes or tubes, and other traditional and typical fluid handling components all well known in the art, arranged to deliver the fluid from the source of fluid to the nozzles 44. The nozzles 44 are distributed around the spray gantry 34 positioned to dispense spray 42 in a vertical and/or transverse direction. The spray gantry 34 comprises control means for sequentially altering the composition of spray 42 to be soap solution, rinse solution, or disinfectant. Control means for sequentially altering the composition of spray 42 comprises a computer or other programmable controller controlling at least one valve in communication with said at least one source of fluid. Control means such as these are well known in the art, and may be programmed as described below. Various embodiments of spray 42 could use high-pressure, low-pressure, foam, or combinations thereof. Optionally, different compositions of spray 42 can be discharged out of separate corresponding sets of nozzles 44.

Mounted near the second end 4 is a dryer 32. The dryer 32 may be a conventional forced air blower that delivers a continuous sheet of high-velocity air 14 to blow the water off of the shopping carts 6 as they exit the wash tunnel 1. Such dryers 32 are often called air knives and are well known in the art. Optionally, the continuous sheet of high-velocity air 14 may be heated for faster drying. In an alternate embodiment, dryer 32 may be ducted into the wash tunnel to dry the shopping carts 6 before they are conveyed out of the wash tunnel 1.

The spray gantry 34 is programmed to operate in a wash sequence. First, an operator places shopping carts 6 in the wash tunnel 1 through the first end 2. Upon activation, the spray gantry 34 moves along the gantry tracks 36 from the start position 38 to the return position 40 while spray 42 is dispensing soap solution. Upon reaching the return position 40, the spray gantry 34 may switch the composition of spray 42 to a rinse solution, which may be water in this embodiment. The spray gantry 34 moves from the return position 40 to the start position 38 while spray 42 discharges rinse solution. Upon reaching the start position 38, the spray gantry 34 may change the composition of spray 42 to a disinfectant. The spray gantry 34 then moves from the start position 38 to the return position 40 while spray 42 is dispensing disinfectant. Disinfectant spray 42 can be any disinfecting or sanitizing agent suitable for spray application. Alternate embodiments may omit dispensing a disinfectant, or may change the order in which the fluids are dispensed. When the spray gantry 34 reaches the return position 40 after dispensing disinfectant, the spray gantry 34 can return to the start position 38. Vent 30 may be provided to remove fumes from the wash tunnel 1 for safe application of the disinfectant. An operator then pushes the shopping carts 6 out through the second end 4 while the dryer 32 is activated. In alternate embodiments, automated means for conveying the plurality of shopping carts can be employed to move carts into and out of the wash tunnel 1, such as conveyor 16 or other devices as discussed above. Optionally, the carts can enter the wash tunnel 1 and exit the wash tunnel 1 through the first end 2, in which case the dryer 32 is mounted near the first end 2. In further embodiments of the cart sanitizing system, spray 42 is steam.

The system disclosed in the second embodiment may be used with a method of sanitizing shopping carts. The method comprises placing shopping carts 6 in a wash tunnel 1, and programming a spray gantry 34 to pass repeatedly over the carts dispensing a spray 42, where in the first pass spray 42 comprises a soap solution, in the second pass spray 42 comprises a rinse solution, and in a third pass, spray 42 comprises a disinfectant; and passing the shopping carts 6 under a continuous sheet of high-velocity air 14 upon exit from the wash tunnel 1.

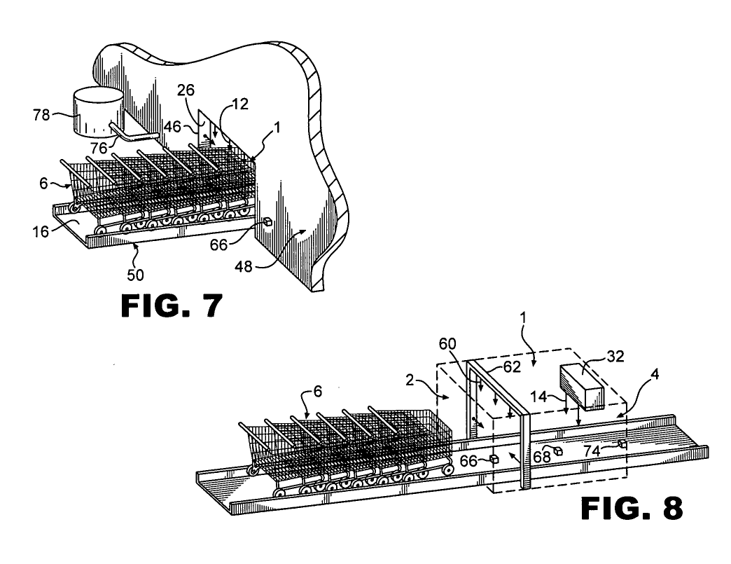

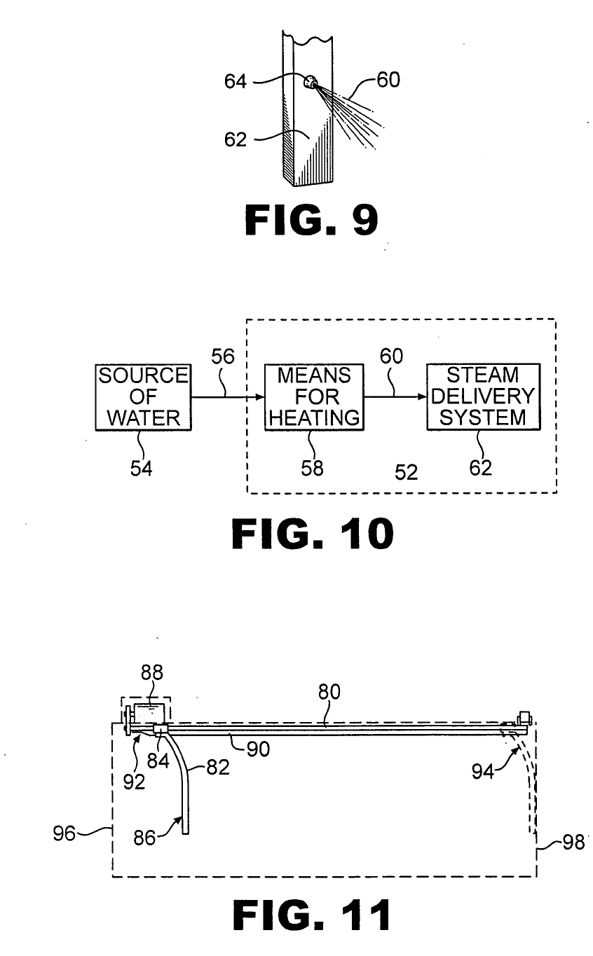

A third embodiment of the cart sanitizing system is shown in FIG. 7. The wash tunnel 1 is installed in a cart entrance 46 of a retail building 48. The wash tunnel 1 comprises the disinfectant delivery apparatus 26 that dispenses disinfectant spray 12 through one or more nozzles 28, as illustrated in FIG. 4. Shopping carts 6 pass through the disinfectant spray 12 as they enter the retail building 48. In one embodiment, cart moving means 50 are employed to keep the cart operator away from the disinfectant spray 12. The cart moving means 50 utilize a conveyor 16 in this embodiment, but any means for conveying a plurality of shopping carts could readily be adapted, as discussed above.

It is contemplated that the third embodiment through the wall of a retail building could employ the first embodiment or the second embodiment of the present invention. Alternate embodiments could dispense additional sprays, such as a rinse and/or dry.

The system disclosed in the third embodiment may be used with a method of sanitizing shopping carts. The method comprises propelling a plurality of shopping carts 6 through a wash tunnel 1 and dispensing a disinfectant spray 12 over the shopping carts 6 in the wash tunnel 1.

A fourth embodiment of the cart sanitizing system is shown in FIG. 8. The cart sanitizing system includes a wash tunnel 1 with a first end 2 and a second end 4. The wash tunnel 1 is sized to accommodate a plurality of shopping carts 6 that enter through the first end 4 of the wash tunnel 1. The wash tunnel 1 includes a steam cleaning apparatus 52, well known in the art. The steam cleaning apparatus 52 is in communication with a source of water 54 that provides water 56 to the steam cleaning apparatus 52. The water 56 passes through a means of heating 58 where it is heated to a temperature higher than the boiling point of the water 56. In one embodiment, the means of heating 58 is an electric or gas heat exchanger designed to raise the temperature of the water 56 to higher than the boiling point of the water 56. At the boiling point of the water 56, the water 56 undergoes a phase change into steam 60. A steam delivery system 62 is in communication with the means of heating 58, and the steam 60 passes into the steam delivery system 62. The steam delivery system 62 includes one or more nozzles 64 to direct the steam 60 onto the shopping carts 6. In one embodiment, the steam 60 is at an elevated pressure such that the steam 60 exiting the nozzles 64 impinges onto the shopping carts 6 with force. In an alternate embodiment, the steam 60 exits the nozzles 64 at or near atmospheric pressure onto the shopping carts 6.

The cart sanitizing system utilizing a steam cleaning apparatus 52 can employ conveyor 16, or any other means for conveying a plurality of shopping carts into and/or out of the wash tunnel as described above. The shopping carts 6 can exit the wash tunnel 1 by the first end 2 or the second end 4. In one embodiment as shown in FIG. 11, the means for conveying a plurality of shopping carts includes a continuous screw shaft 80 rotatably supported in the wash tunnel 1 above the shopping carts 6. The continuous screw shaft 80 extends from the first end 2 to the second end 4. The means for conveying a plurality of shopping carts further comprises a push arm 82, having a driven end 84 and a pushing end 86. The continuous screw shaft 80 comprises a screw thread along the length of the continuous screw shaft 80, operably connected to corresponding features on the driven end 84. A motor 88 is drivingly connected to the continuous screw shaft 80, whereby the screw thread advances the push arm 82 along the continuous screw shaft 80 when the continuous screw shaft 80 rotates. In one embodiment, the corresponding features on the driven end 84 include an internal thread that matches the thread of the continuous screw shaft 80. Alternately, the driven end 84 may operably connect to the continuous screw shaft 80 using any protrusion that engages the thread of the continuous screw shaft 80. A control system allows an operator to start and stop the motor 88. A track 90 is installed adjacent to the continuous screw shaft 80 to orient the push arm 82 as it travels between a home position 92 at the first end 2, and a stop position 94 at the second end 4. When the push arm 82 is at the home position 92, the track holds the push arm 82 in a horizontal position so that the shopping carts can pass under the push arm 82. When the motor 88 rotates the continuous screw shaft 80, the push arm 82 begins to travel along the screw thread of the continuous screw shaft 80. When the push arm 82 leaves its home position 92, the track 90 allows the push arm 82 to rotate down into a vertical orientation, wherein the pushing end 86 is positioned to push against the shopping cart closest to the first end 2. As the push arm 82 travels from the first end 2 to the second end 4, the pushing end 86 pushes said first cart, which in turn pushes the shopping carts through the wash tunnel 1 and out through the second end 4. When the push arm 82 reaches the stop position 94, the motor 88 rotates in the opposite direction, causing the push arm 82 to translate along the continuous screw shaft 80 in a reverse direction from the stop position 94 to the home position 92.

In one embodiment, a dryer 32 is mounted in the wash tunnel 1 to dry the shopping carts 6 as the shopping carts 6 exit the wash tunnel 1. The dryer 32 may be a conventional forced air blower that delivers a continuous sheet of high-velocity air 14 to blow the water off of the shopping carts 6. Such dryers 32 are often called air knives and are well known in the art. In another embodiment, the dryer 32 is ducted into the wash tunnel 1 to fill the wash tunnel 1 with a large volume of drying air before the shopping carts are conveyed out.

In some embodiments, it may be desirable to equip the wash tunnel 1 with doors for safety or other reasons. As illustrated in FIG. 11, a first door 96 substantially covers the first end, and a second door 98 substantially covers the second end. In one embodiment, the first door 96 and second door 98 lock during the wash cycle and/or when the wash tunnel 1 is not in use.

The system disclosed in the fourth embodiment may be used with a method of sanitizing shopping carts. The method comprises placing a plurality of shopping carts 6 in a wash tunnel 1 and then dispensing steam 60 over the shopping carts 6.

The presence of shopping carts 6 in the cart sanitizing system may be monitored. By monitoring the presence of shopping carts 6, the cart sanitizing system can be deactivated when shopping carts 6 are not present. By coordinating the operation of the cart sanitizing system to the presence of shopping carts 6, energy costs and chemical use can be minimized. Sensing means for detecting the position of the shopping carts may include one or more proximity sensors 66 to monitor the presence or position of a shopping cart 6. Means for detecting the position of the shopping carts will herein be referenced as proximity sensors 66, but may include any sensor suitable for detecting the presence of the shopping carts, including but not limited to devices deploying radio frequency, light, acoustic, photoelectric, laser, magnetic, or electronic sensors, all of which are well known in the art. Sensing means for detecting the position of the shopping carts also includes physical contact switches and other physical sensing devices, which are well known in the art.

In the first embodiment as illustrated in FIG. 1, the proximity sensors 66 can be positioned at the first end 2, between the soap spray 8 and the rinse spray 10, between the rinse spray 12 and the disinfectant spray 14, between the disinfectant spray 14 and the dryer 32, and at the second end 4. As the shopping carts 6 are conveyed through the wash tunnel 1, the soap spray 8 activates when one or more shopping carts 6 pass the first proximity sensor 66, and continues until the shopping carts 6 pass a second proximity sensor 68. Likewise, the rinse spray 10 activates when the shopping carts 6 pass the second proximity sensor 68, and continues until the shopping carts 6 pass a third proximity sensor 70. The disinfectant spray 12 activates when the shopping carts 6 pass the third proximity sensor 70, and continues until the shopping carts 6 pass a fourth proximity sensor 72. Finally, the dryer 32 activates when the shopping carts 6 pass the fourth proximity sensor 72, and continues until the shopping carts 6 pass a fifth proximity sensor 74. Alternately, a timer could be coupled with the proximity sensor 66 to allow the cart sanitation system to activate for a sufficient duration for a shopping cart 6 to pass through the wash tunnel 1 given the uniform speed of the conveyor 16.

In the second embodiment as illustrated in FIG. 5, a proximity sensor 66 may monitor the presence of one or more shopping carts 6. The spray 42 would not activate when no shopping cart 6 is present to conserve water and chemicals.

In the third embodiment as illustrated in FIG. 7, a proximity sensor 66 may monitor the presence of one or more shopping carts 6. In order to conserve disinfectant, the disinfectant spray 12 can be programmed to activate only when the conveyor 16 is moving and the proximity sensor 66 senses the presence of the shopping carts 6.

In the fourth embodiment as illustrated in FIG. 1, the proximity sensors 66 can be positioned at the first end 2 before the steam delivery system 62, between the steam delivery system 62 and the dryer 32, and at the second end 4. The steam 60 activates when one or more shopping carts 6 pass the first proximity sensor 66 and continues until the shopping carts pass the second proximity sensor 68. The dryer 32 activates when the shopping carts 6 pass the second proximity sensor 68, and continues until the shopping carts 6 pass a third proximity sensor 74.

The cart sanitizing system dispenses chemicals or steam, and for environmental reasons the fluid dispensed by the system and the effluent from the shopping carts should be contained. The cart sanitizing system may provide a means for reclaiming fluid, comprising a means for diverting waste fluids 76 and a waste fluid reclamation unit 78. The means for diverting waste fluids 76 comprises drains, valves, pumps, pipes or tubes, and other traditional and typical fluid handling components, all well known in the art, arranged to deliver the dispensed fluid and effluent from the wash tunnel 1 to the waste fluid reclamation unit 78. The waste fluid reclamation unit 78 may be a collection and filtration system similar to those used in car wash or industrial water reclamation units well known in the art. In one embodiment, the waste fluid reclamation unit 78 treats the effluent and recycles it in the cart sanitizing system. Alternately, the waste fluid reclamation unit 78 collects the waste for proper disposal.

The cart sanitizing system can be embodied in a mobile unit for commercial cleaning services. In a mobile embodiment, the wash tunnel 1 is mounted onto a truck or trailer for convenient transporting to retail locations for cart cleaning. In a mobile unit, the conveyor 16 may be divided into sections for compact transporting.

The present invention provides retailers with a cart sanitation system that is so convenient and effective that it can be used to sanitize shopping carts every day. The cart sanitation system operates efficiently by washing many carts at one time and by reclaiming and recycling waste fluids. By employing the cart sanitation system, the spread of diseases via shopping carts can be reduced and the public health will benefit.

Additional advantages and modifications will readily occur to those skilled in the art. Therefore, the invention in its broader aspects is not limited to the specific details and illustrative examples shown and described herein. Accordingly, various modifications may be made without departing from the scope of the general inventive concept disclosed herein.

What is claimed is:

1. An apparatus for cleaning shopping carts comprising:
a. a longitudinally-extending tunnel having an entrance and an exit, wherein the tunnel is adapted to house shopping carts;
b. means for conveying the shopping carts through the tunnel from the entrance to the exit;
c. means for detecting the position of the shopping carts when the shopping carts are positioned within the tunnel between the entrance and the exit;
d. an array of nozzles housed within the tunnel and connected to at least one source of fluid; and
e. means for controlling the array of nozzles;
wherein the controlling means are in communication with the detecting means;
and wherein the fluid emanates from the array of nozzles when the detecting means detect the shopping carts within the tunnel.

2. The apparatus of claim 1, wherein the fluid is steam, soap, water, or disinfectant.

3. The apparatus of claim 1, wherein the array of nozzles comprises:
a. a first array of nozzles located near the entrance, wherein the first array of nozzles is connected to a first fluid,
b. a second array of nozzles spaced from the first array of nozzles, wherein the second array of nozzles is connected to a second fluid; and
c. a third array of nozzles located near the exit, wherein the third array of nozzles is connected to a third fluid.

4. The apparatus of claim 3, wherein the first fluid is soap, the second fluid is water, and the third fluid is disinfectant.

5. The apparatus of claim 4, further comprising a dryer located near the exit.

6. The apparatus of claim 5, further comprising:
means for reclaiming at least one of the first fluid, second fluid, and third fluid, wherein the means for reclaiming fluid collect the emanated fluid from within the tunnel for disposal.

7. The apparatus of claim 1, further comprising:
a. one or more gantry tracks positioned longitudinally within the tunnel between the entrance and the exit, the gantry tracks having a start position and a stop position; and
b. a gantry adapted for traveling along the gantry track in a first direction toward the stop position and a second direction toward the start position, wherein the array of nozzles is affixed to the gantry.

8. The apparatus of claim 7, wherein the array of nozzles emanates a first fluid while the gantry is traveling along the track in the first direction, and a second fluid while the gantry is traveling along the track in the second direction.

9. The apparatus of claim 8, further comprising a dryer located near the exit.

10. The apparatus of claim 2, wherein the tunnel is installed through the wall of a building, so that the entrance is located outside the building and the exit is located inside the building.

11. An apparatus for cleaning shopping carts comprising:
a. a tunnel having a longitudinal axis, an entrance, and an exit, wherein the tunnel is adapted to house one or more shopping carts;
b. at least one array of nozzles housed within the tunnel and connected to at least one source of fluid;
c. means for controlling the at least one array of nozzles, and
d. means for conveying the shopping carts along the longitudinal axis from the entrance to the exit, comprising:
a continuous screw shaft rotatably supported by the tunnel parallel to the longitudinal axis between the entrance and the exit, the continuous screw shaft adapted to rotate above the shopping carts;
a motor drivingly connected to the continuous screw shaft;
a push arm, having a driven end and a pushing end, the driven end being operably connected to the continuous screw shaft, and the pushing end being positioned to engage at least one of said one or more shopping carts near the entrance; and
control means for selectively actuating the motor,
whereby the rotating continuous screw shaft advances the push arm along the continuous screw shaft toward the exit, wherein the push arm pushes the shopping carts out of the exit.

12. The apparatus of claim 11, further comprising:
means for detecting the position of the shopping carts when the shopping carts are positioned within the tunnel between the entrance and the exit,
wherein the means for controlling the array of nozzles are in communication with the means for detecting the position of the shopping carts;
and wherein the fluid emanates from the array of nozzles when the detecting means detect the position of the shopping carts within the tunnel.

13. The apparatus of claim 12, further comprising:
a. a first door substantially covering the entrance; and
b. a second door substantially covering the exit.

14. The apparatus of claim 13, wherein the fluid is steam.

15. The apparatus of claim 14, further comprising a blower, wherein the blower directs air onto each of the at least one shopping cart.

16. The apparatus of claim 15, further comprising:
means for reclaiming fluid, wherein the means for reclaiming fluid collect condensate and the emanated fluid from within the tunnel for disposal.

17. An apparatus for cleaning shopping carts comprising:
a tunnel having a longitudinal axis, an entrance at a first end, and an exit, wherein the tunnel is adapted to house one or more shopping carts;
a first door substantially covering the entrance and a second door substantially covering the exit;
a continuous screw shaft rotatably supported by the tunnel parallel to the longitudinal axis between the entrance and the exit, the continuous screw shaft adapted to rotate above the shopping carts; a motor drivingly connected to the continuous screw shaft; control means for selectively actuating the motor; a push arm, having a driven end and a pushing end, the driven end being operably connected to the continuous screw shaft, whereby the rotating continuous screw shaft advances the push arm along the continuous screw shaft toward the exit; the pushing end being oriented to engage at least one of said one or more shopping carts, whereby the motion of the push arm along the continuous screw shaft pushes the shopping carts out of the exit;
means for detecting the position of the shopping carts when the shopping carts are within the tunnel;
an array of nozzles housed within the tunnel and connected to a source of steam, wherein steam emanates from the array of nozzles onto the shopping carts when the detecting means detect the position of the shopping carts within the tunnel.

18. The apparatus of claim 17, further comprising:
means for reclaiming fluid, wherein the means for reclaiming fluid collect condensate and the emanated fluid from within the tunnel for disposal.

19. The apparatus of claim 18, further comprising a blower, wherein the blower directs air onto the shopping carts.
